(19) United States
(12) Patent Application Publication
(10) Pub. No.: US 2026/0063645 A1
(43) Pub. Date: Mar. 5, 2026

(54) BIOMARKER COMPOSITIONS, PROTEIN PANELS, DEVICES, AND METHODS TO IDENTIFY RISK OF AND TREAT PULMONARY VASCULAR INADEQUACY IN SINGLE VENTRICLE HEART DISEASE

(71) Applicant: The Regents of the University of Colorado, A Body Corporate, Denver, CO (US)

(72) Inventors: Benjamin Frank, Denver, CO (US); Jesse Davidson, Denver, CO (US)

(21) Appl. No.: 19/318,194

(22) Filed: Sep. 3, 2025

Related U.S. Application Data
(60) Provisional application No. 63/690,197, filed on Sep. 3, 2024.

(51) Int. Cl. G01N 33/68

(52) U.S. Cl. CPC G01N 33/6893; G01N 2333/50; G01N 2333/96494; G01N 2800/324; G01N 2800/50

(57) ABSTRACT

Disclosed herein are methods, compositions and devices for diagnosing pathologic pulmonary vascular disease development, progression, and outcomes from cardiac surgical interventions, particularly surgical palliation in SVHD, and more particularly superior cavopulmonary anastomosis procedures. In particular, methods are provided for assessing risk of post-Stage 2 complications arising from intolerance of Stage 2 physiology due to e.g., pulmonary vascular inadequacy.

### CROSS-REFERENCE TO RELATED APPLICATIONS

This application claims priority to U.S. Provisional Patent Application No. 63/690,197, entitled “BIOMARKER COMPOSITIONS, PROTEIN PANELS, DEVICES, AND METHODS TO IDENTIFY RISK OF AND TREAT PULMONARY VASCULAR INADEQUACY IN SINGLE VENTRICLE HEART DISEASE”, filed on Sep. 3, 2024, the contents of which are hereby incorporated herein in their entirety.

### GOVERNMENT RIGHTS

This invention was made with Government support under NIH/NHLBI K23HL163490, NIH/NCATS Colorado CTSA, No. ULI TR001082, and NIH/NHLBI K23HL123634 awarded by the National Institutes of Health. The Government has certain rights in the invention.

### BACKGROUND

Single ventricle heart disease (SVHD) is a severe form of congenital heart disease (CHD) affecting between 2 and 8 in 10,000 live births in the United States in which patients are born with a single functional pumping chamber. While there is no cure for SVHD, with staged surgical palliation many affected children can survive into adulthood. Beginning in the first week after birth (following a Stage 1 palliation for many patients) and continuing to the Stage 2 operation, the interstage period is a crucial time for children with SVHD. During interstage, children have an unobstructed pathway from the single ventricle to the systemic circulation and a pressure-limiting source of flow to the pulmonary circulation-most often a shunt between the aorta or right ventricle and the pulmonary arteries. The Stage 2 operation that ends the interstage period-achieved via a Glenn or hemi-Fontan anastomosis—is typically performed at 4-6 months of age. In this operation, the patient's mechanism of blood flow to the lungs is surgically converted from an active process driven by ventricular systole to a passive process via a direct connection of the superior vena cava to the pulmonary arteries. This abrupt change makes the peri-Stage 2 period the optimal time to evaluate both pre-operative markers of interstage pulmonary vascular growth and the post-operative sufficiency of the pulmonary vasculature to accept passive blood flow.

Among children who undergo Stage 2 palliation, up to 25% experience a complication in the immediate post-operative period that is directly linked to pulmonary vascular disease causing impaired pulmonary blood flow. These include severe hypoxemia, respiratory failure, and persistent pleural effusions. Despite this high clinical burden, there are no validated metrics or biomarkers available to reliably identify SVHD patients at particular risk for pulmonary vascular inadequacy. Diagnosis and treatment of pathologic pulmonary vascular development are therefore limited due to poor understanding of the underlying mechanisms.

### SUMMARY

Disclosed herein is a method of assessing risk of a morbidity to surgical palliation in a subject having single-ventricle heart disease (SVHD), which can include: obtaining a sample of circulating proteins from the subject prior to the surgical palliation, testing the sample to determine a level of one or more protein biomarkers selected from the group consisting of matrix metalloproteinase (MMP), tissue inhibitor of metalloproteinase (TIMP), and fibroblast growth factor (FGF), comparing the level to a reference range for the one or more protein biomarkers, and identifying the subject as being at risk for the morbidity where the level is outside the reference range.

In some embodiments, the method can also include where the surgical palliation includes creating a cavopulmonary anastomosis in the subject. The method can also include where the one or more protein biomarkers are selected from FGF 3, FGF 4, FGF 5, FGF 6, FGF 9, FGF 12, FGF 17, FGF 18, FGF 20, FGF 22, FGF 23, FGFR1, FGFR2, FGFBP1, MMP 1, MMP 2, MMP 7, MMP 8, MMP 10, MMP 13, MMP 14, MMP 16, MMP 17, MMP 20, TIMP 1, TIMP 2, and TIMP 4. The method can also include where the one or more protein biomarkers are selected from FGF 3, FGF 4, FGF 5, FGF 6, FGF 9, FGF 12, FGF 17, FGF 18, FGF 20, FGF 23, FGFR1, FGFBP1, MMP 2, MMP 7, MMP 8, MMP 13, MMP 16, MMP 17, MMP 20, TIMP 1, and TIMP 2. The method can also include where the one or more protein biomarkers comprise MMP 7 and MMP 8.

The method can also include where the morbidity includes an increase in a post-operative variable selected from: percentage of first 48 post-operative hours with hypoxemia, endotracheal intubation time, and length of stay (LOS) as compared to patients having a circulating level within the reference range. In some embodiments, the morbidity can include an increase in percentage of first 48 post-operative hours with hypoxemia, and the one or more protein biomarkers are selected from MMP 7, MMP 8, MMP 14, MMP 17, FGFR2, and FGF 22, or more particularly can include MMP 7, MMP 8, MMP 17, and FGFR2. The method may also include where the one or more protein biomarkers include one of MMP 7 or MMP 17, the level of which is above the reference range. The method can also include where the one or more protein biomarkers include one of MMP 8 or FGFR2, the level of which is below the reference range. In some embodiments, the morbidity can include LOS, and the one or more protein biomarkers are selected from MMP 1, MMP 7, MMP 8, MMP 10, TIMP 4, and FGF 23, or more particularly can include MMP 1, MMP 7, MMP 8, and TIMP 4. The method can also include where the one or more protein biomarkers include one of MMP 8 or MMP 1, the level of which is below the reference range. The method can also include where the one or more protein biomarkers include one of MMP 7 or TIMP 4, the level of which is above the reference range. The method can also include changing a circulating level of a protein biomarker in the subject prior to the surgical palliation.

Disclosed herein is a composition for diagnosing pulmonary vascular inadequacy in a subject having single-ventricle heart disease (SVHD), which can include at least one molecule having affinity for a biomarker, where the biomarker is a protein selected from group consisting of matrix metalloproteinase (MMP), tissue inhibitor of metalloproteinase (TIMP), and fibroblast growth factor (FGF). In some embodiments, the composition can also include where the biomarker is one or more of FGF 3, FGF 4, FGF 5, FGF 6, FGF 9, FGF 12, FGF 17, FGF 18, FGF 20, FGF 22, FGF 23, FGFR1, FGFR2, FGFBP1, MMP 1, MMP 2, MMP 7, MMP 8, MMP 10, MMP 13, MMP 14, MMP 16, MMP 17, MMP 20, TIMP 1, TIMP 2, and TIMP 4. The composition can also include where the biomarker is one or more of FGF 3, FGF 4, FGF 5, FGF 6, FGF 9, FGF 12, FGF 17, FGF 18, FGF 20, FGF 23, FGFR1, FGFBP1, MMP 2, MMP 7, MMP 8, MMP 13, MMP 16, MMP 17, MMP 20, TIMP 1, and TIMP 2. The composition can also include the molecule is selected from a nucleic acid, a peptide, or combinations thereof. Also disclosed herein is a device which can include a surface configured to bind the molecule.

### BRIEF DESCRIPTION OF THE DRAWINGS

FIG. 1 shows a diagram of the study described herein. From the original single ventricle cohort of 73, three subjects were excluded from analysis due to inability to confirm heparin status at the time of sampling. Four additional subjects were excluded from the post-operation analysis due to undergoing a non-Stage 2 operation after enrollment.

FIG. 2A shows graphs of the results of an SVHD case-control analysis. Box plots depict adjusted log-abundance for proteins that differed between non-heparinized cases and non-heparinized controls. MMP=matrix metalloprotease, FGF=fibroblast growth factor.

FIG. 2B shows additional graphs of results of the SVHD case-control analysis. TIMP=tissue inhibitor of metalloprotease.

FIG. 3 presents an analysis of the association between measured proteins and hypoxemia burden. Scatterplots depict the log transformed, center scaled, heparin-status adjusted protein abundance and post-Stage 2 hypoxemia burden for individual study subjects. Best fit line and 95% confidence interval are overlaid to depict the relationship between the variables. MMP=matrix metalloprotease, FGF=fibroblast growth factor.

FIG. 4 presents an analysis of the association between proteins and length of stay. Scatterplots depict the log transformed, center scaled, heparin-status adjusted protein abundance and post-Stage 2 length of stay for individual study subjects. Best fit line and 95% confidence interval are overlaid to depict the relationship between the variables. MMP=matrix metalloprotease, FGF=fibroblast growth factor, TIMP=tissue inhibitor of metalloprotease.

### DETAILED DESCRIPTION

Disclosed herein are methods, compositions and devices for diagnosing pathologic pulmonary vascular disease development, progression, and outcomes from cardiac surgical interventions, particularly surgical palliation in SVHD, and more particularly superior cavo-pulmonary anastomosis procedures. In particular methods are provided for assessing risk of post-Stage 2 complications arising from intolerance of Stage 2 physiology due to e.g., pulmonary vascular inadequacy.

Using targeted cardiovascular proteomic profiling of infants undergoing Stage 2 palliation, Applicants have demonstrated a distinct serum proteomic phenotype among SVHD infants who manifest pulmonary vascular inadequacy compared to those with better pulmonary blood flow. Through an evaluation of circulating levels of a multitude of protein families, Applicants identified members of the matrix metalloproteinase (MMP), tissue inhibitor of metalloproteinase (TIMP), and fibroblast growth factor (FGF) families as potentially important in the distinct proteomic fingerprints identified. The MMPs and their inhibitors, the TIMPs, are primary endogenous modulators of extracellular matrix production and activity of several members has been linked to cardiac and pulmonary pathology in a variety of populations. The FGFs are a heterogenous family of growth factors that includes several members with activity tied to angiogenesis and fibrosis. No prior work has taken a comprehensive, targeted approach to mapping the circulating levels of any of these protein families in the SVHD population.

Applicants conducted a prospective, cohort study of targeted mapping of the circulating MMP, TIMP, and FGF protein families in SVHD infants undergoing Stage 2 palliation. Applicants hypothesized that infants with SVHD would have different circulating levels of multiple measured proteins compared to healthy controls and that a panel containing a subset of the MMP, TIMP, and FGF proteins would be able to predict patients at greater risk for post-Stage 2 complications associated with insufficient pulmonary blood flow.

Applicants disclose that targeted, family-level mapping identified broad alterations in circulating MMPs, TIMPs, and FGFs in interstage infants with SVHD compared to healthy controls. After adjusting for clinical covariates, single biomarker testing identified several targets significantly associated with either post-Stage 2 hypoxemia or post-operative length of stay (LOS), including FGF 22, FGF 23, FGFR2, MMP 1, MMP 7, MMP 8, MMP 10, MMP 14, MMP 17, and TIMP 4. Multivariable models accounting for key clinical and demographic variables identified increased circulating MMP 7 and decreased circulating MMP 8 as significantly associated with both hypoxemia and LOS. These findings indicate that dysregulation of extracellular matrix production, and specifically imbalance of MMP 7 and MMP 8, can be an important driver of post-Stage 2 morbidity in SVHD.

The study cohort included subjects age 31 days to 2 years with SVHD either undergoing pre-Stage 2 catheterization or Stage 2 palliation without plans for cardiac catheterization for enrollment. Subjects were preferably selected prior to pre-Stage 2 catheterization in cases where catheterization was deemed clinically necessary. Applicants considered any form of superior cavo-pulmonary anastomosis (Glenn or Hemi-Fontan operation) as a Stage 2 independent of whether a subject had previously undergone a Stage 1 procedure. Applicants excluded subjects with a so-called 1.5 ventricle repair (those with a persistent, additional pulsatile source of pulmonary blood flow). Control subject inclusion criteria were subjects greater than 4 kg, 3-12 months of age, undergoing anesthesia for an elective, non-cardiac procedure with a plan for a clinically indicated peripheral IV. Applicants excluded control subjects having known or suspected cardiac, pulmonary, infectious, or genetic abnormalities.

The reported clinical data were extracted from electronic medical records including cardiac diagnosis and dominant ventricle morphology, medication regimen, catheterization results, and hospitalization duration. Post-operative oxygen saturations were extracted at 1-minute intervals from the BedMaster™ hemodynamic monitoring system in the cardiac intensive care unit (Anandic Medical Systems, Feuerthalen, Switzerland). Study data were collected and managed using REDCap electronic data capture tools hosted at the University of Colorado. Applicants identified a priori clinical variables to evaluate the relationship between protein biomarkers of interest and subjects with more or less favorable clinical outcomes. Primary variables of interest included: percent of time in the first 48 post-operative hours with clinically significant hypoxemia, defined as an oxygen saturation below 70% (also referred to herein as “48 h Low Sat %”); and LOS. Secondary variables included: pre-Stage 2 pulmonary vascular resistance and post-Stage 2 endotracheal intubation time (ETT); chest tube days; and volume of chest tube drainage.

All pre-operation whole blood samples were obtained under general anesthesia. Samples were collected in the early morning after a period of fasting for most patients. For SVHD subjects enrolled at a pre-Stage 2 catheterization, systemic vein samples were collected prior to any procedural interventions. For subjects enrolled at other times, a systemic venous sample was obtained on the day of Stage 2 palliation before initiation of cardiopulmonary bypass. Control subject samples were obtained from a systemic vein after induction of anesthesia at the time of IV placement. All samples were processed for serum at the time of collection, aliquoted, and stored at −80° C. until batch analysis.

Samples underwent targeted proteomic phenotyping using an aptamer-based assay. The detailed methods of this assay and prior validation data have been previously published (Gold L, Ayers D, Bertino J et al. Aptamer-based multiplexed proteomic technology for biomarker discovery. PLOS One 2010). Briefly, each protein target has its own binding reagent comprised of fluorescent tag-labeled modified DNA, referred to as a modified aptamer. Each serum sample was incubated with a mixture of the modified aptamers generating aptamer-protein complexes. After elimination of unbound aptamers, the bound aptamers were then quantified using a hybridization array. Quality control confirmed intra-assay and inter-assay variability of <5%. Raw abundance measurements were log-transformed, centered to mean 0 and scaled to variance 1. Prior to assay, Applicants a priori identified 39 candidate proteins of interest for targeted analysis based on membership in the MMP, TIMP, and FGF families, or status as FGF-interacting: MMP 1-3, 7-10, 12-14, 16-17, 19-20, TIMP 1-4, FGF 1-10, 12, 16-20, 22-23, FGFR1, FGFR2, and FGF P1. For simplicity, both true members of the FGF family and its associated proteins FGFR1, FGFR2, and FGF P1 are referred to herein as “FGFs”.

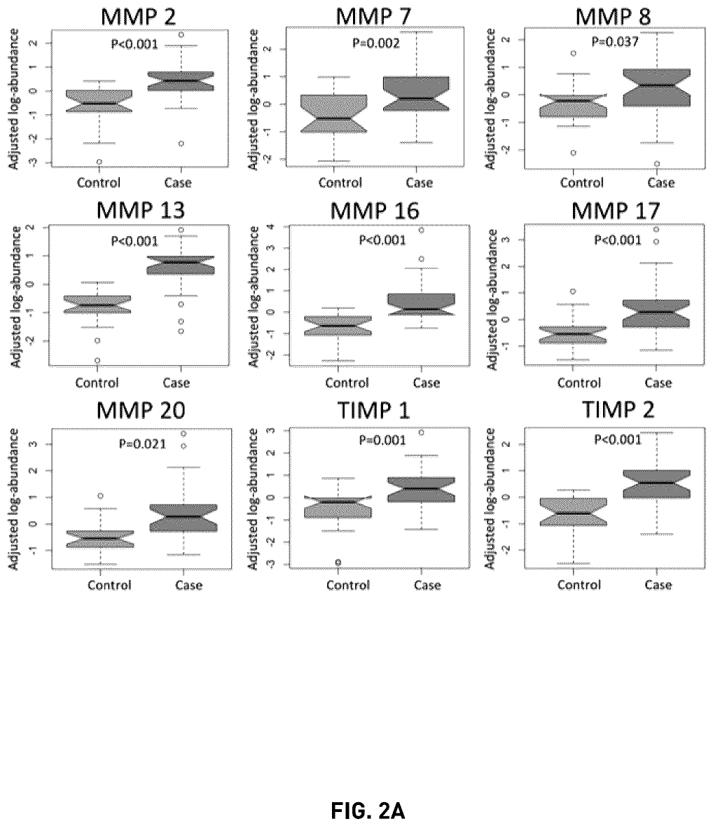

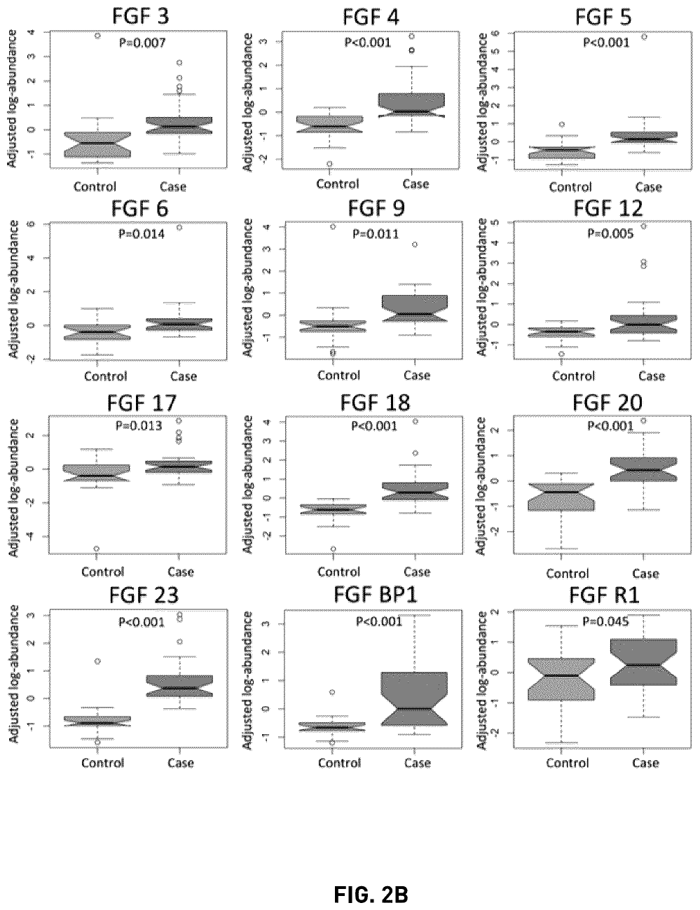

Applicants evaluated whether circulating levels of any members of the MMP, TIMP, or FGF protein families were associated with SVHD case-control status. Applicants identified 21 proteins with different circulating abundances between infants with SVHD and controls (FIG. 2A, FIG. 2B). In some embodiments, biomarkers of interstage SVHD can include FGF 3, FGF 4, FGF 5, FGF 6, FGF 9, FGF 12, FGF 17, FGF 18, FGF 20, FGF 23, FGFR1, FGFBP1, MMP 2, MMP 7, MMP 8, MMP 13, MMP 16, MMP 17, MMP 20, TIMP 1, and TIMP 2.

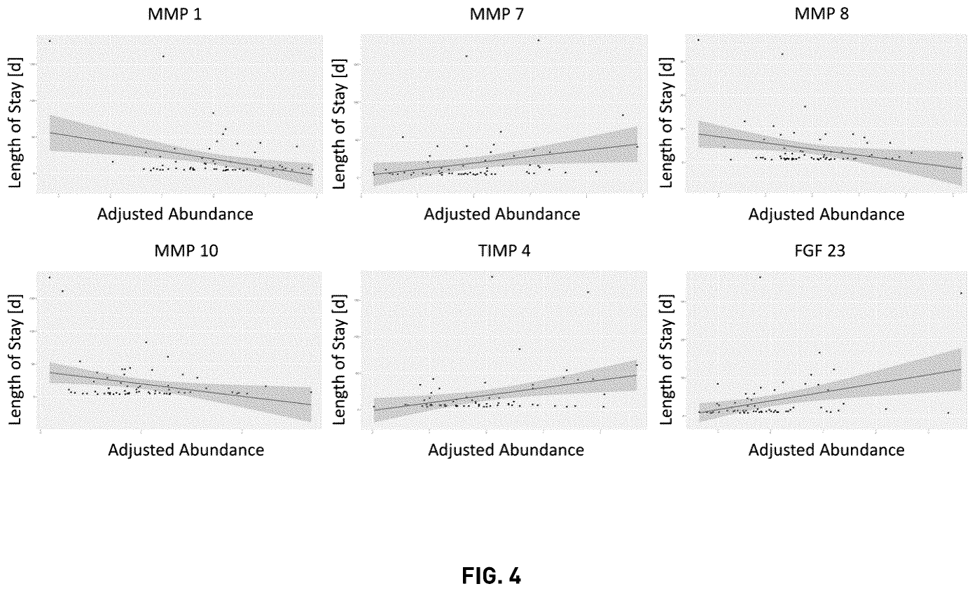

Applicants disclose that abundances of MMP, TIMP, and FGF proteins are associated with post-operation outcomes in SVHD patients, including percentage of first 48 post-operative hours with saturation <70% (48 h low sat %), endotracheal intubation time, and LOS. Applicants found that, after controlling for clinical covariates, six proteins showed a significant linear association with post-operation hypoxemia burden: MMP 7, MMP 8, MMP 14, MMP 17, FGFR2, and FGF 22. Applicants disclose that a model including four protein abundances (MMP 7, MMP 8, MMP 17, and FGFR2) in addition to growth index demonstrated a moderate ability to predict 48 h low sat % (Table 3). MMP 7, MMP 17, and growth index levels were higher in subjects with a greater hypoxemia burden while MMP 8 and FGFR2 levels were lower in those who experienced more hypoxemia. Applicants further found that, after controlling for clinical covariates, six proteins showed a significant linear association with LOS: MMP 1, MMP 7, MMP 8, MMP 10, TIMP 4, and FGF 23 (FIG. 4). Applicants disclose that a model including abundance of MMP 1, MMP 7, MMP 8, and TIMP 4 with age, sex, ventricular morphology and growth index demonstrated a moderate ability to predict LOS (Table 3). MMP 7 and TIMP 4 levels were higher in subjects with longer LOS, while MMP 1 and MMP 8 levels were lower.

These findings indicate an association between extracellular matrix dysregulation and adverse post-Stage 2 outcomes in SVHD. Significant changes were observed in 50% of the measured MMP and TIMP family members in SVHD cases compared to healthy controls; for 19 of 20 altered proteins, Applicants found higher levels in the SVHD cohort than in the healthy children. The MMP family of proteins and their endogenous inhibitors, the TIMPs, arc the major modulators of extracellular matrix composition in a variety of tissues. Increased MMP activity has been demonstrated to be pathologic in the pulmonary vasculature of both animal and human subjects with pulmonary hypertension (PH). MMP 2 in particular is known to promote intimal hyperplasia and vascular remodeling in PH patients. TIMP 1 levels are higher among adults with PH compared to healthy controls and TIMP 2 has shown an association with pulmonary arterial stiffness in children with PH. Increased levels of multiple MMPs and TIMPs in the cohort (including MMP 2, TIMP 1, and TIMP 2) may therefore, indicate increased extracellular matrix turnover, a pathologic change that could put interstage SVHD patients at risk for adverse pulmonary vascular outcomes.

The circulating concentration of 11 of the 21 tested circulating FGFs differed significantly between cases and controls, with higher levels seen among cases for all proteins. The FGFs are a functionally diverse group of 18 circulating growth factors, grouped into 6 subfamilies based on sequence homology and function, with important effects mediated through a variety of tissue-specific FGF receptors (FGFRs) ranging from angiogenesis to neurodevelopment and maintenance of homeostasis. The 3 FGFs that were most significantly upregulated in SVHD cases compared to controls were FGF 4, 20, and 23. FGF 4 is a pro-angiogenic factor that has been studied as potentially protective in adults with insufficient coronary blood flow. Without being bound to a particular theory, higher levels among SVHD patients may therefore represent an adaptive response to improve sub-optimal tissue-level perfusion secondary to interstage physiology. FGF 20 has been linked to anti-inflammatory properties in intestinal tissues; its roles in the heart and lung are less well defined. Finally, high levels of circulating FGF 23 are known to contribute to airway inflammation, endothelial dysfunction, endogenous nitric oxide dysregulation, myocardial hypertrophy, and cognitive dysfunction. Applicants' work has demonstrated that post-Stage 2 circulating FGF 23 is one of the major drivers of the hypoxemic fingerprint. As discussed above, Applicants observed both elevated interstage FGF 23 in SVHD compared to controls and a linear association between pre-Stage 2 FGF 23 level and longer post-Stage 2 LOS within the SVHD infants. Put together, these findings suggest that elevated FGF 23 in SVHD both pre- and post-Stage 2 can be maladaptive and place patients at risk for intolerance of Stage 2 physiology.

In the study, two proteins in particular were associated with both adverse outcomes of interest: increased MMP 7 and decreased MMP 8. Mechanistically, increased MMP 7 activity is believed to worsen both parenchymal lung disease and pulmonary vascular disease through promotion of endothelial to mesenchymal transition, increased fibrosis, and increased vascular smooth muscle cell proliferation. Applicants' work has additionally demonstrated an association between increased post-operative MMP 7 and post-Stage 2 hypoxemia. Increased circulating MMP 7 is also associated with pulmonary pathology in a number of clinical populations, including idiopathic pulmonary fibrosis, chronic obstructive pulmonary disease, and pulmonary arterial hypertension. In this context, the results described herein suggest a role for increased MMP 7 exposure as both a biomarker of disease and a potential mechanistic driver of pulmonary vascular inadequacy in SVHD.

Circulating levels of MMP 8, while elevated in SVHD compared to controls, showed an inverse association with both LOS and post-operative hypoxemia burden within the SVHD cohort. The association between higher MMP 8 levels and more favorable outcomes is consistent with a role for MMP 8 as pro-angiogenic and supportive of endothelial cell function. Further, MMP 8 may be protective, opposing progression of pulmonary vascular disease in adult pulmonary hypertension patients. The combination of elevated MMP 8 in SVHD compared to controls but an association between higher MMP 8 and more favorable outcomes suggests that failure to activate MMP 8 can be a mechanistic driver of pulmonary vascular inadequacy in SHVD patients. Collectively, Applicants' findings indicate that an imbalance between the proliferative effects of MMP 7 and the antiproliferative effects of MMP 8 can be deleterious in interstage SVHD patients.

Two additional biomarkers of interest, TIMP 4 and MMP 1, were directly associated with post-Stage 2 LOS. While the role of MMP 1 in the pulmonary vasculature is less clear, elevated TIMP 4 level has been identified as a biomarker of disease and indicator of poor prognosis in pulmonary arterial hypertension. Although not evaluated previously in SVHD, elevated TIMP 4 levels are linked to increased pulmonary vascular stiffness by MRI, providing a potential mechanism linking TIMP 4 activity to impaired tolerance of Stage 2 physiology.

In various embodiments, upregulated biomarkers may display greater than about 1× protein expression, for example greater than about 1.5×, 2×, 3×, 4×, 5×, 6×, 7×, 8×, 9×, 10×, 20×, 30×, 40×, 50×, 60×, 70×, 80×, 90× or 100×, whereas downregulated biomarkers may display expression that is more than about 1× lower than normalized protein expression, for example decreased by more than about 1.5×, 2×, 3×, 4×, 5×, 6×, 7×, 8×, 9×, 10×, 20×, 30×, 40×, 50×, 60×, 70×, 80×, 90× or 100×. In some embodiments, a level of biomarker may be compared to a reference range that includes a level found in subjects not having SVHD or some otherwise identified control group. Protein biomarkers and molecules recognizing same, as disclosed herein, may be full-length or fragments thereof.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

Affinity may refer to the avidity of one molecule for another—for example an antibody for a target antigen. In some embodiments, affinity can be presented as a relative binding—e.g. competition of two antibodies for the same target molecule.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Diagnose” or “diagnostic” refers identifying and/or detecting the presence or absence of or nature of a disease or disorder. Such detection methods can be used, for example, for early diagnosis of the condition, to determine whether a subject is predisposed to a disease or disorder, to monitor the progress of the disease or disorder or the progress of treatment protocols, to assess the severity of the disease or disorder, to forecast the an outcome of a disease or disorder and/or prospects of recovery, or to aid in the determination of a suitable treatment for a subject. Disclosed herein are compositions and methods useful in detecting and/or diagnosing various diseases, disorders, and conditions, which may be characterized by one or more symptoms associated with, for example pulmonary vascular inadequacy, and/or single-ventricle heart disease (SVHD), or any of the diseases and conditions disclosed within. In many cases, detecting may involve combining a biomarker with a detecting molecule having affinity for the biomarker. The molecule may then be detected, isolated, identified or analyzed by various methods known to those of skill in the art.

The terms “modulate”, “modulation” and the like refer to the ability of a compound to increase or decrease the function, or activity of an organism, cell, protein, peptide, gene, biomarker, etc (“target”). “Modulation”, in its various forms, is intended to encompass inhibition, antagonism, partial antagonism, activation, agonism and/or partial agonism of the activity associated with the target. Inhibitors compounds may bind to, partially or totally block stimulation, decrease, prevent, delay activation, inactivate, desensitize, or down regulate signal transduction. The ability of a compound to modulate a target's activity can be demonstrated in various ways, such as nucleic acid quantitation (northern analysis), an enzymatic assay or a cell-based assay.

A “patient” or “subject” includes a mammal or animal, such as a human, cow, horse, sheep, lamb, pig, chicken, turkey, quail, cat, dog, mouse, rat, rabbit, or guinea pig. The animal can be a mammal such as a non-primate or a primate (e.g., monkey and human). In one embodiment, a patient is a human, such as a human infant, child, adolescent, or adult of any or indeterminant sex. In most embodiments disclosed herein the subject or patient is a newborn child.

“Prevention” as used herein means the avoidance of the occurrence or of the re-occurrence of a disease, disorder, or condition as specified herein, by the administration of a composition, compound, treatment, or therapy according to the present disclosure to a subject in need thereof.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

Further exemplary embodiments are disclosed below in the Examples and the disclosure are provided and described for all non-limiting purposes.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

### Examples

Presented herein is a prospective, cohort study of targeted mapping of the circulating MMP, TIMP, and FGF protein families in SVHD infants undergoing Stage 2 palliation. Applicants hypothesized that infants with SVHD would have different circulating levels of multiple measured proteins compared to healthy controls and that a panel containing a subset of the MMP, TIMP, and FGF proteins would be able to predict patients at greater risk for post-Stage 2 complications associated with insufficient pulmonary blood flow.

### Methods

### Subjects

The Colorado Multiple Institution Review Board approved this study. Written informed consent was obtained from the study subjects' parents in all cases. In this prospective cohort study, Applicants approached all subjects age 31 days to 2 years at Children's Hospital Colorado in Denver, Colorado, USA from 2018-2021 with SVHD either undergoing pre-Stage 2 catheterization or Stage 2 palliation without plans for cardiac catheterization for enrollment. (11) When possible, subjects were approached prior to their pre-Stage 2 catheterization. For those not approached prior to catheterization this occurred either due to catheterization occurring prior to the research team having an opportunity to obtain informed consent or because a pre-Stage 2 catheterization was not deemed clinically necessary. Applicants considered any form of superior cavo-pulmonary anastomosis (Glenn or Hemi-Fontan operation) as a Stage 2 independent of whether a patient had previously undergone a Stage 1 procedure. Applicants excluded patients with a so-called 1.5 ventricle repair (those with a persistent, additional pulsatile source of pulmonary blood flow).

Control subjects were identified from the main operating room schedule at Children's Hospital Colorado. Control subject inclusion criteria were patients greater than 4 kg, 3-12 months of age, undergoing anesthesia for an elective, non-cardiac procedure with a plan for a clinically indicated peripheral IV. Applicants excluded potential control subjects if they had any known or suspected cardiac, pulmonary, infectious, or genetic abnormalities.

### Clinical Data

The reported clinical data were extracted from the electronic medical record including cardiac diagnosis and dominant ventricle morphology, medication regimen, catheterization results, and hospitalization duration. Post-operative oxygen saturations were extracted at 1-minute intervals from the BedMaster™ hemodynamic monitoring system in the cardiac intensive care unit (Anandic Medical Systems, Feuerthalen, Switzerland). Study data were collected and managed using REDCap electronic data capture tools hosted at the University of Colorado. Applicants identified a priori clinical variables to evaluate the relationship between protein biomarkers of interest and subjects with more or less favorable clinical outcomes. The primary variables of interest were percent of time in the first 48 post-operative hours with clinically significant hypoxemia, defined as an oxygen saturation below 70% (48 h Low Sat %), and post-operative length of stay (LOS). Secondary variables included pre-Stage 2 pulmonary vascular resistance and post-Stage 2 endotracheal intubation time (ETT), chest tube days, and volume of chest tube drainage.

### Sample Collection and Protein Analysis

All pre-operation whole blood samples were obtained under general anesthesia. Samples were collected in the early morning after a period of fasting for most patients. For SVHD subjects enrolled at a pre-Stage 2 catheterization, systemic vein samples were collected prior to any procedural interventions. For subjects enrolled at other times, a systemic venous sample was obtained on the day of Stage 2 palliation before initiation of cardiopulmonary bypass. Control subject samples were obtained from a systemic vein after induction of anesthesia at the time of IV placement. All samples were processed for serum at the time of collection, aliquoted, and stored at −80° C. until batch analysis.

Samples underwent targeted proteomic phenotyping using an aptamer-based assay performed by SomaLogic, Inc. at their laboratory in Boulder, Colorado, USA. The detailed methods of this assay and prior validation data have been previously published. (12) Briefly, each protein target has its own binding reagent comprised of fluorescent tag-labeled modified DNA, referred to as a modified aptamer. Each serum sample was incubated with a mixture of the modified aptamers generating aptamer-protein complexes. After elimination of unbound aptamers, the bound aptamers were then quantified using a hybridization array. Quality control confirmed intra-assay and inter-assay variability of <5%. Raw abundance measurements were log-transformed, centered to mean 0 and scaled to variance 1. Higher expression values correspond to higher protein levels but are not an absolute quantification of protein concentrations. Prior to assay, Applicants a priori identified 39 candidate proteins of interest for targeted analysis based on membership in the MMP, TIMP, and FGF families, or status as FGF-interacting: MMP 1-3, 7-10, 12-14, 16-17, 19-20, TIMP 1-4, FGF 1-10, 12, 16-20, 22-23, FGFR1, FGFR2, and FGFBP1. For simplicity, Applicants have referred to both true members of the FGF family and its associated proteins FGFR1, FGFR2, and FGFBP1 as “FGFs”.

### Statistical Analysis

Demographic and clinical variables were summarized using descriptive statistics as indicated by the distribution of the data. Statistical analyses were performed in R. The Wilcoxon rank sum test was used to compare continuous variables and Fisher's exact testing was used for categorical variables. Analysis of the distribution of the protein abundance data revealed that whether a patient received heparin prior to sample acquisition affected the level of several proteins of interest. Therefore, Applicants performed the case-control analysis in 2 ways. First, Applicants considered all SVHD cases and healthy controls using multiple linear regression for each protein target (n=39) to regress out the heparin effect. In this analysis, protein abundance was the outcome of interest and case-control status was a candidate predictor adjusted for sex and heparin status. In a second analysis, Applicants directly compared the protein abundances of the subset of cases who did not receive heparin to those of the controls (none of whom received heparin).

For the post-operative clinical outcome analysis only the SVHD cases were considered. The a priori primary outcomes of interest were 48 h low sat % and post-operative LOS. In this analysis Applicants first accounted for the effect of heparin status on each individual protein abundance for each subject using a one-way ANOVA model fit. The residuals from these 39 model fits were then considered as adjusted protein abundances and used for all downstream analyses. Applicants then fit protein-by-protein linear regression models (n=39) with covariates including adjusted protein abundance, age, sex, ventricular morphology, and growth index (weight in kg/height in m). Proteins with a significant association with the outcome of interest (p<0.05 in the model) were considered for multivariable analysis. To identify the optimal model for predicting the outcomes of interest Applicants considered all possible combinations of the variables identified using all subsets regression. Applicants considered adjusted R², Mallow's Cₚ, and Bayesian Information Criterion (BIC) as best subset model selection criteria. (13-15) Among the three models (one selected based on each of the 3 model selection criteria) Applicants performed five-fold cross validation repeated 100 times and selected the winner model based on the one with the smallest average root mean-squared error. Alpha value to determine significance was set at 0.05 for all analyses.

### Results

### Study Population

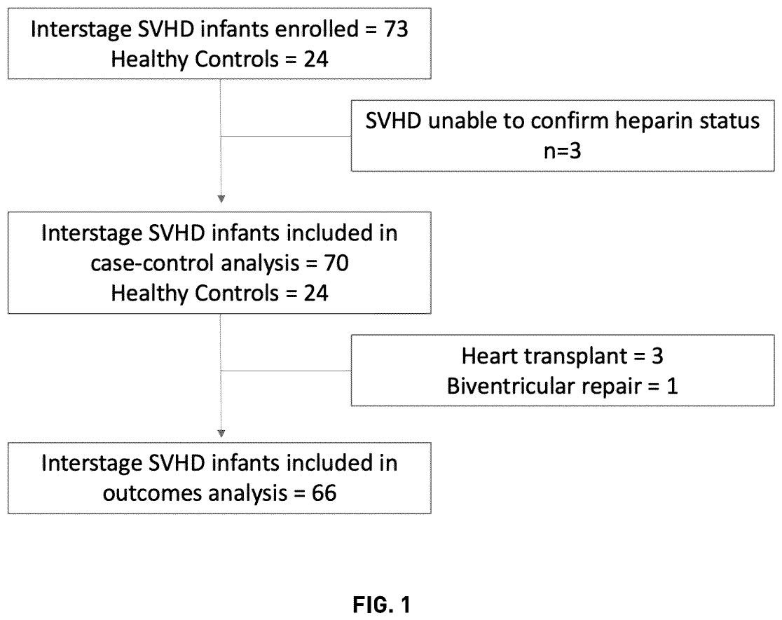

Seventy-three SVHD cases and twenty-four similar age healthy controls were enrolled (FIG. 1). Pre-operation systemic vein samples were available for all cases and controls. Thirty-seven cases were found to have received a dose of heparin prior to their sample being drawn, while 33 cases and all 24 controls had no heparin exposure. In 3 subjects Applicants were unable to confirm the timing of heparin administration relative to sample collection; those subjects were excluded from further analysis. Therefore, the final pre-operation analysis included n=70 SVHD cases and n=24 controls. Four SVHD subjects ultimately did not undergo Stage 2 palliation (3 heart transplantation, 1 biventricular repair), so 66 SVHD subjects were included in the post-operation outcomes analysis. Control subjects were undergoing surgery with urology (e.g. hypospadias, chordee), orthopedics (e.g. supernumerary digit, hip dysplasia), neurosurgery (e.g. craniosynostosis), ophthalmology (e.g. strabismus), or other surgical teams (e.g. inguinal hernia). Demographics and clinical characteristics are presented in Table 1.

TABLE 1

| | Control | SVHD | |
|---|---|---|---|
| | (n = 24) | (n = 70) | p-value |
| Weight (kg) | 7.995 | 5.700 | 1.384E−10 |
| | (6.580, 10.380) | (3.800, 10.400) | |
| Height (cm) | 69.10 | 61.75 | 1.084E−09 |
| | (63.50, 80) | (41, 87) | |
| Growth Index (kg/m) | 11.69 | 9.38 | 8.60E−10 |
| | (10.14, 12.97) | (7.31, 13.42) | |
| Age (days) | 248.5 | 124 | 7.806E−12 |
| | (156, 355) | (26, 649) | |
| Sex (F) | 10 | 30 | 1 |
| | (41.67%) | (42.86%) | |
| Heparin EXPOSURE | 0 | 37 | 9.704E−07 |
| (Y) | (0%) | (52.86%) | |
| Mean PA pressure | | 13 | |
| (mmHg) | | (8, 35) | |
| PVRI (UNITS*M²) | | 2 | |
| | | (0.70, 14) | |
| Qp (l/min/M²) | | 3.60 | |
| | | (1.40, 11.60) | |
| Qs (l/min/M²) | | 3.18 | |
| | | (1.92, 8.33) | |
| Qp/Qs | | 1.10 | |
| | | (0.30, 3.80) | |
| System ventricle | | 6.50 | |
| EDP (mmHg) | | (3, 14) | |
| Mean SPO2 for the | | 78.60 | |
| first 48 hours (%) | | (67.60, 86.80) | |
| 48 H LOW SAT % | | 4.20 | |
| (%) | | (0.10, 62.60) | |
| Discharge PDE 5i | | 22 | |
| therapy (Yes) | | (31.43%) | |
| Post-op length of | | 7 | |
| stay (days) | | (4, 182) | |
| Endotracheal Intubation | | 16 | |
| duration (hours) | | (0, 504) | |
| Chest tube duration | | 2 | |
| (days) | | (1, 15) | |
| Total pleural drainage | | 137 | |
| (ml) | | (0, 701) | |

Abbreviations: SVHD = single ventricle heart disease, PA = pulmonary artery, PVRi = indexed pulmonary vascular resistance, Qp = pulmonary blood flow, Qs = systemic blood flow, EDP = end diastolic pressure, SpO2 = oxygen saturation, 48 h low sat % = percent time in the first 48 post-operative hours with oxygen saturation <70%, PDE 5i = phosphodiesterase type 5 inhibitor. Variables expressed as median (range) or n (%).

Table 1. Abbreviations: SVHD=single ventricle heart disease, PA=pulmonary artery, PVRi=indexed pulmonary vascular resistance, Qp=pulmonary blood flow, Qs=systemic blood flow, EDP=end diastolic pressure, SpO2=oxygen saturation, 48 h low sat %=percent time in the first 48 post-operative hours with oxygen saturation <70%, PDE 5i=phosphodiesterase type 5 inhibitor. Variables expressed as median (range) or n (%).

### Pre-Operation Differences Between Cases and Controls

Applicants first considered all SVHD cases and evaluated whether circulating levels of any members of the MMP, TIMP, or FGF protein families were associated with case-control status after adjusting for patient sex and heparin status. Case-control status was associated with 20 of the 39 tested proteins, including 7 of 14 MMPs, 2 of 4 TIMPs, and 11 of 21 FGFs (Table 2). Measured abundances were higher in cases than controls for all significant proteins except MMP 20. In the second case-control analysis, considering only the subgroup of cases who did not receive heparin, Applicants identified 21 proteins with different circulating abundances between infants with SVHD and controls (FIG. 2A and FIG. 2B). Notably, the same 20 proteins identified in the heparin-adjusted case-control analysis were again identified in the non-heparin exposed subgroup analysis, with one additional protein noted in the second set (FGF 17).

TABLE 2
Multiple Linear Regression Analysis of SVHD Cases versus
Controls Proteins whole circulating abundance differs between
cases and controls after adjusting for sex and heparin
status. Positive coefficients indicate higher abundance
in SVHD cases than controls whereas negative coefficients
indicate higher protein abundance in controls.

| PROTEIN | Coefficient | P-valuE |
|---|---|---|
| FGF 23 | 1.30E+00 | 8.60E−09 |
| MMP 13 | 1.02E+00 | 8.04E−08 |
| MMP 16 | 9.41E−01 | 9.43E−08 |
| FGF 20 | 8.64E−01 | 7.65E−07 |
| TIMP 2 | 1.08E+00 | 1.52E−06 |
| FGF 4 | 7.37E−01 | 2.37E−05 |
| MMP 2 | 9.45E−01 | 3.76E−05 |
| FGFBP1 | 8.07E−01 | 3.97E−05 |
| FGF 5 | 0.810707 | 0.000147 |
| MMP 17 | 0.589192 | 0.001133 |
| MMP 7 | 0.833692 | 0.001717 |
| FGF 12 | 0.714738 | 0.00247 |
| FGF 18 | 0.491514 | 0.00397 |
| FGF 3 | 0.570472 | 0.005341 |
| FGF 6 | 0.628252 | 0.005475 |
| MMP 20 | −0.48239 | 0.017481 |
| FGF 9 | 0.562842 | 0.018628 |
| TIMP 1 | 0.58405 | 0.024967 |
| FGFR1 | 0.548051 | 0.03642 |
| MMP 8 | 0.540144 | 0.040557 |

Abbreviations: SVHD = single ventricle heart disease, MMP = matrix metalloprotease, FGF = fibroblast growth factor, TIMP = tissue inhibitor of metalloprotease.

Table 2: Multiple Linear Regression Analysis of SVHD Cases versus Controls Proteins whole circulating abundance differs between cases and controls after adjusting for sex and heparin status. Positive coefficients indicate higher abundance in SVHD cases than controls whereas negative coefficients indicate higher protein abundance in controls. Abbreviations: SVHD=single ventricle heart disease, MMP=matrix metalloprotease, FGF=fibroblast growth factor, TIMP=tissue inhibitor of metalloprotease.

### Post-Operative Outcomes

The SVHD study population experienced moderate morbidity in the post-Stage 2 period (Table 1). The median patient spent 4.2% of the first 48 post-operative hours with saturation <70%. The distribution of 48 h low sat % was right skewed such that the mean was 11.9% and the range of observed outcomes varied from 0.1% to 62.6%. Similarly, while the median endotracheal intubation time was 16 hours, the range of observed outcomes varied from 0 to 504 hours. No patient required an emergent return to the operating room and all patients survived to discharge from the hospital. Twenty-two subjects were discharged home on targeted pulmonary hypertension therapy (phosphodiesterase type 5 inhibitors in all cases). Median length of stay for the entire SVHD cohort was 7 days (range 4-182 days), and all patients were discharged on supplemental oxygen due to residence at high elevation, per local protocol.

### Association Between Protein Abundances and Post-Operation Outcomes

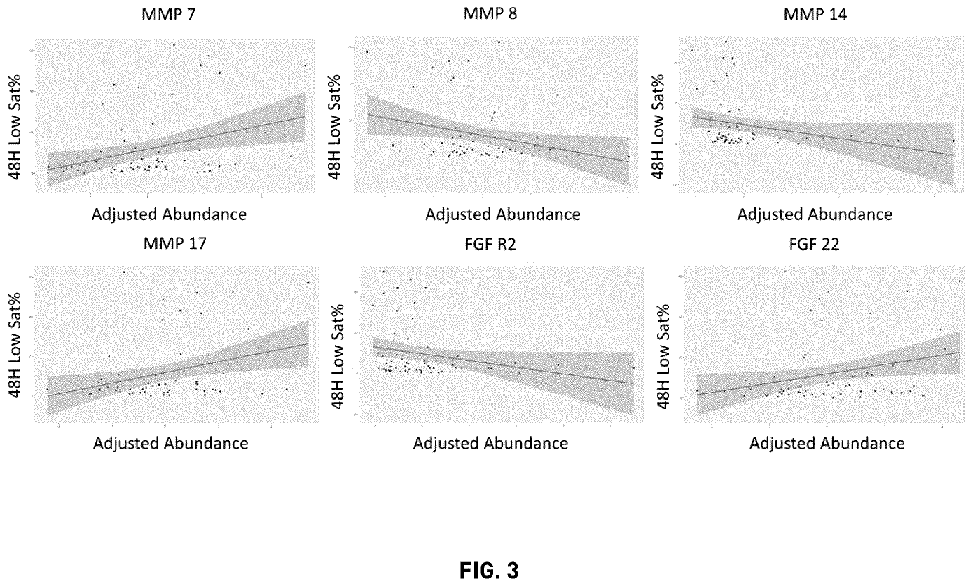

Applicants next evaluated whether any of the individual protein abundances were associated with the primary outcome of interest, 48 h low sat %. After controlling for clinical covariates, testing the protein abundances one-by-one there were six that showed a significant linear association with post-operation hypoxemia burden: MMP 7, MMP 8, MMP 14, MMP 17, FGFR2, and FGF 22. Scatterplots depicting the relationship between each adjusted circulating protein abundance and 48 h low sat % for the individual patients are shown in FIG. 3 (Pearson correlation coefficient and p-value for each scatterplot-MMP 7:0.3462, 0.0072; MMP 8: −0.2865, 0.0278; MMP 14: −0.2562, 0.0502; MMP 17:0.3272, 0.0114; FGFR2: −0.2474, 0.0589; FGF 22:0.2778, 0.0332). These six proteins along with age, sex, ventricular morphology, and growth index were then considered for the multivariable analysis. The final selected model included four protein abundances (MMP 7, MMP 8, MMP 17, and FGFR2) in addition to growth index and demonstrated a moderate ability to predict the outcome variable (R²=0.416, adjusted R²=0.361; Table 3). MMP 7, MMP 17, and growth index levels were higher in subjects with a greater hypoxemia burden while MMP 8 and FGFR2 levels were lower in those who experienced more hypoxemia.

| | | |
|---|---|---|
| Protein | Coefficient | P value |

48 h LOW SAT % adjusted r² = 0.361

| | | |
|---|---|---|
| MMP 7 | 7.07 | 4.96E−04 |
| MMP 17 | 5.53 | 3.33E−03 |
| MMP 8 | −3.79 | 3.72E−02 |
| FGFR2 | −3.51 | 5.79E−02 |
| Growth index | 4.64 | 1.52E−02 |

Length of Stay adjusted r² = 0.363

| | | |
|---|---|---|
| MMP 7 | 7.63 | 2.16E−02 |
| MMP 8 | −7.3 | 2.71E−02 |
| MMP 1 | −12.31 | 4.43E−04 |
| TIMP 4 | 12.51 | 3.30E−04 |

Table 3: Results of Multivariable Models Evaluating the Relationship Between Serum Protein Abundances and Outcomes of Interest. Proteins and clinical covariates associated with post-operative hypoxemia burden (top) and length of stay (bottom) in the final selected multivariable model. Considered clinical covariates included age, sex, ventricular morphology, and growth index. Positive coefficients indicate a direct relationship between the candidate predictor and outcome of interest while negative coefficients indicate an inverse relationship. Abbreviations: MMP=matrix metalloprotease, FGF=fibroblast growth factor, 48 h low sat %=percent of the first 48 post-operative hours with oxygen saturation <70%.

Finally, Applicants turned attention to post-operation LOS. After controlling for clinical covariates, testing the protein abundances one by one there were six that showed a significant linear association with LOS: MMP 1, MMP 7, MMP 8, MMP 10, TIMP 4, and FGF 23. Scatterplots depicting the relationship between each adjusted circulating protein abundance and LOS for the individual patients are shown in FIG. 4 (Pearson correlation coefficient and p-value for each scatterplot—MMP 1: −0.3611, 0.0031; MMP 7: 0.2704, 0.0294; MMP 8: −0.2905, 0.0189; MMP 10: −0.2992, 0.0155; TIMP 4: 0.3291, 0.0074; FGF 23: 0.3617, 0.0031). These six proteins, along with age, sex, ventricular morphology, and growth index were considered for the multivariable analysis. The final selected model demonstrated a moderate ability to predict the outcome variable (R^{2=0.403}, adjusted R^{2=0.363}) and included four protein abundances (MMP 1, MMP 7, MMP 8, and TIMP 4; Table 3). MMP 7 and TIMP 4 levels were higher in subjects with longer LOS, while MMP 1 and MMP 8 levels were lower.

### DISCUSSION

Applicants present a prospective, cohort study of comprehensive mapping of circulating MMP, TIMP, and FGF protein family members in interstage infants with SVHD and healthy controls. Applicants report (1) significant differences between cases and controls among multiple members of all 3 protein families including 20 of 39 targeted proteins, (2) a significant association between increased interstage levels of MMP 7, MMP 17, and FGF 22, and decreased levels of MMP 8, MMP 14, and FGFR2 and post-Stage 2 hypoxemia burden after controlling for clinical covariates, and (3) a significant association between increased interstage levels of MMP 7, TIMP 4, and FGF 23, and decreased levels of MMP 1, MMP 8, and MMP 10 and post-Stage 2 length of stay after controlling for clinical covariates. Multivariable modeling demonstrated that, after accounting for clinical covariates, MMP 7, MMP 8, MMP 17, and FGFR2 had a persistent relationship with hypoxemia while MMP 1, MMP 7, MMP 8, and TIMP 4 had a persistent relationship with LOS. These findings indicate an association between extracellular matrix dysregulation and adverse post-Stage 2 outcomes in SVHD.

MMP, TIMP, and FGF dysregulation in interstage SVHD

In the cohort Applicants noted significant changes in 50% of the measured MMP and TIMP family members in SVHD cases compared to healthy controls; for 19 of the 20 altered proteins, Applicants found higher levels in the SVHD cohort than in the healthy children. The MMP family of proteins and their endogenous inhibitors, the TIMPs, are the major modulators of extracellular matrix composition in a variety of tissues. Increased MMP activity has been demonstrated to be pathologic in the pulmonary vasculature of both animal and human subjects with pulmonary hypertension (PH). MMP 2 in particular is known to promote intimal hyperplasia and vascular remodeling in PH patients. TIMP 1 levels are higher among adults with PH compared to healthy controls and TIMP 2 has shown an association with pulmonary arterial stiffness in children with PH. Increased levels of multiple MMPs and TIMPs in the cohort (including MMP 2, TIMP 1, and TIMP 2) may therefore, indicate increased extracellular matrix turnover, a pathologic change that could put interstage SVHD patients at risk for adverse pulmonary vascular outcomes.

The circulating concentration of 11 of the 21 tested circulating FGFs differed significantly between cases and controls, with higher levels seen among cases for all proteins. The FGFs are a functionally diverse group of 18 circulating growth factors, grouped into 6 subfamilies based on sequence homology and function, with important effects mediated through a variety of tissue-specific FGF receptors (FGFRs) ranging from angiogenesis to neurodevelopment and maintenance of homeostasis. The 3 FGFs that were most significantly upregulated in SVHD cases compared to controls were FGF 4, 20, and 23. FGF 4 is a pro-angiogenic factor that has been studied as potentially protective in adults with insufficient coronary blood flow. Higher levels among SVHD patients, therefore, may represent an adaptive response to improve sub-optimal tissue-level perfusion secondary to interstage physiology. FGF 20 has been linked to anti-inflammatory properties in intestinal tissues; its roles in the heart and lung are less well defined. Finally, high levels of circulating FGF 23 are known to contribute to airway inflammation, endothelial dysfunction, endogenous nitric oxide dysregulation, myocardial hypertrophy, and cognitive dysfunction. Applicants' work has demonstrated that post-Stage 2 circulating FGF 23 is one of the major drivers of the hypoxemic fingerprint. On single biomarker testing, controlling for clinical covariates, in this study Applicants found both elevated interstage FGF 23 in SVHD compared to controls and a linear association between pre-Stage 2 FGF 23 level and longer post-Stage 2 LOS within the SVHD infants. Put together, these findings suggest that elevated FGF 23 in SVHD both pre- and post-Stage 2 could be maladaptive and place patients at risk for intolerance of Stage 2 physiology. Future, mechanistic studies evaluating the cellular and tissue level effects of FGF dysregulation in SVHD will be important.

### Association Between Biomarkers and Adverse Outcomes

On single variable testing Applicants found significant associations between pre-Stage 2 levels of multiple biomarkers of interest and each of the primary outcomes. Two proteins in particular were associated with both adverse outcomes of interest: increased MMP 7 and decreased MMP 8. Mechanistically, increased MMP 7 activity is thought to worsen both parenchymal lung disease and pulmonary vascular disease through promotion of endothelial to mesenchymal transition, increased fibrosis, and increased vascular smooth muscle cell proliferation. Applicants have additionally demonstrated an association between increased post-operative MMP 7 and post-Stage 2 hypoxemia. Increased circulating MMP 7 has also been associated with pulmonary pathology in a number of clinical populations, including idiopathic pulmonary fibrosis, chronic obstructive pulmonary disease, and pulmonary arterial hypertension. In this context, these results suggest a role for increased MMP 7 exposure as both a biomarker of disease and a potential mechanistic driver of pulmonary vascular inadequacy in SVHD.

Circulating levels of MMP 8, while elevated in SVHD compared to controls, showed an inverse association with both length of stay and post-operative hypoxemia burden within the SVHD cohort. The association between higher MMP 8 levels and more favorable outcomes is aligned with prior mechanistic work suggesting a role for MMP 8 as pro-angiogenic and supportive of endothelial cell function. Further, a recent translational article demonstrated that MMP 8 may be protective, opposing progression of pulmonary vascular disease in adult pulmonary hypertension patients. The combination of elevated MMP 8 in SVHD compared to controls but an association between higher MMP 8 and more favorable outcomes suggests that failure to activate MMP 8 in a subset of the SVHD cohort may be a mechanistic driver of pulmonary vascular inadequacy. Collectively, these findings raise the possibility that an imbalance between the proliferative effects of MMP 7 and the antiproliferative effects of MMP 8 may be deleterious in interstage SVHD patients and is an important area for future mechanistic study.

Two additional biomarkers of interest, TIMP 4 and MMP 1, were directly associated with post-Stage 2 LOS. While the role of MMP 1 in the pulmonary vasculature is less clear, elevated TIMP 4 level has been previously identified as a biomarker of disease and indicator of poor prognosis in pulmonary arterial hypertension. Although not evaluated previously in SVHD, a pediatric study linked elevated TIMP 4 levels to increased pulmonary vascular stiffness by MRI providing a potential mechanism linking TIMP 4 activity to impaired tolerance of Stage 2 physiology.

Applicants did not find evidence for a significant association between age at Stage 2, sex, or ventricular morphology and either outcome, a difference from some prior studies that have suggested prolonged LOS in younger patients undergoing Stage 2. Applicants did note an unexpected association between higher growth index (weight per height) and post-operative hypoxemia. As prior studies have shown smaller weight rather than larger weight to be a risk factor for adverse outcomes, this association may be driven by linear growth faltering in a subset of the SVHD cohort. Further studies specifically targeting detailed assessment of growth parameters in interstage SVHD will be important.

What is claimed is:

1. A method of assessing risk of a morbidity to surgical palliation in a subject having single-ventricle heart disease (SVHD), comprising:
obtaining a sample of circulating proteins from the subject prior to the surgical palliation;
testing the sample to determine a level of one or more protein biomarkers selected from the group consisting of matrix metalloproteinase (MMP), tissue inhibitor of metalloproteinase (TIMP), and fibroblast growth factor (FGF);
comparing the level to a reference range for the one or more protein biomarkers; and
identifying the subject as being at risk for the morbidity where the level is outside the reference range.

2. The method of claim 1, wherein the surgical palliation comprises creating a cavopulmonary anastomosis in the subject.

3. The method of claim 1, wherein the one or more protein biomarkers are selected from FGF 3, FGF 4, FGF 5, FGF 6, FGF 9, FGF 12, FGF 17, FGF 18, FGF 20, FGF 22, FGF 23, FGFR1, FGFR2, FGFBP1, MMP 1, MMP 2, MMP 7, MMP 8, MMP 10, MMP 13, MMP 14, MMP 16, MMP 17, MMP 20, TIMP 1, TIMP 2, and TIMP 4.

4. The method of claim 1, wherein the one or more protein biomarkers are selected from FGF 3, FGF 4, FGF 5, FGF 6, FGF 9, FGF 12, FGF 17, FGF 18, FGF 20, FGF 23, FGFR1, FGFBP1, MMP 2, MMP 7, MMP 8, MMP 13, MMP 16, MMP 17, MMP 20, TIMP 1, and TIMP 2.

5. The method of claim 1, wherein the one or more protein biomarkers comprise MMP 7 and MMP 8.

6. The method of claim 1, wherein the morbidity comprises an increase in a post-operative variable selected from: percentage of first 48 post-operative hours with hypoxemia, endotracheal intubation time, and length of stay (LOS) as compared to patients having a circulating level within the reference range.

7. The method of claim 6, wherein the morbidity comprises an increase in percentage of first 48 post-operative hours with hypoxemia, and the one or more protein biomarkers are selected from MMP 7, MMP 8, MMP 14, MMP 17, FGFR2, and FGF 22.

8. The method of claim 6, the one or more protein biomarkers comprise MMP 7, MMP 8, MMP 17, and FGFR2.

9. The method of claim 7, wherein the one or more protein biomarkers include one of MMP 7 or MMP 17, the level of which is above the reference range.

10. The method of claim 7, wherein the one or more protein biomarkers include one of MMP 8 or FGFR2, the level of which is below the reference range.

11. The method of claim 6, wherein the morbidity comprises LOS, and the one or more protein biomarkers are selected from MMP 1, MMP 7, MMP 8, MMP 10, TIMP 4, and FGF 23.

12. The method of claim 11, wherein the one or more protein biomarkers comprise MMP 1, MMP 7, MMP 8, and TIMP 4.

13. The method of claim 11, wherein the one or more protein biomarkers include one of MMP 8 or MMP 1, the level of which is below the reference range.

14. The method of claim 11, wherein the one or more protein biomarkers include one of MMP 7 or TIMP 4, the level of which is above the reference range.

15. The method of claim 1, further comprising changing a circulating level of a protein biomarker in the subject prior to the surgical palliation.

16. A composition for diagnosing pulmonary vascular inadequacy in a subject having single-ventricle heart disease (SVHD), comprising at least one molecule having affinity for a biomarker, wherein the biomarker is a protein selected from group consisting of matrix metalloproteinase (MMP), tissue inhibitor of metalloproteinase (TIMP), and fibroblast growth factor (FGF).

17. The composition of claim 16, wherein the biomarker is one or more of FGF 3, FGF 4, FGF 5, FGF 6, FGF 9, FGF 12, FGF 17, FGF 18, FGF 20, FGF 22, FGF 23, FGFR1, FGFR2, FGFBP1, MMP 1, MMP 2, MMP 7, MMP 8, MMP 10, MMP 13, MMP 14, MMP 16, MMP 17, MMP 20, TIMP 1, TIMP 2, and TIMP 4.

18. The composition of claim 16, wherein the biomarker is one or more of FGF 3, FGF 4, FGF 5, FGF 6, FGF 9, FGF 12, FGF 17, FGF 18, FGF 20, FGF 23, FGFR1, FGFBP1, MMP 2, MMP 7, MMP 8, MMP 13, MMP 16, MMP 17, MMP 20, TIMP 1, and TIMP 2.

19. The composition of claim 16, the molecule is selected from a nucleic acid, a peptide, or combinations thereof.

20. A device comprising a surface configured to bind the molecule of claim 16.
